(19) United States
(12) Patent Application Publication
(10) Pub. No.: US 2018/0221193 A1
(43) Pub. Date: Aug. 9, 2018

(54) Penis Enhancement System

(71) Applicant: Philip Vanderwolk, Santa Fe, NM (US)

(72) Inventor: Philip Vanderwolk, Santa Fe, NM (US)

(21) Appl. No.: 15/942,822

(22) Filed: Apr. 2, 2018

Related U.S. Application Data
(63) Continuation-in-part of application No. 14/843,466, filed on Sep. 2, 2015, now Pat. No. 9,962,281.
(60) Provisional application No. 62/045,113, filed on Sep. 3, 2014.
(60) Provisional application No. 62/558,617, filed on Sep. 14, 2017.

(51) Int. Cl. A61F 5/41

(52) U.S. Cl. CPC A61F 5/41; A61F 2005/414

(57) ABSTRACT

A penis enhancement system. The penis enhancement system includes a first and second ring, each having a bead affixed thereto. A cord lock is slidably disposed over each of the first and second rings, wherein the cord lock can adjust a diameter of an opening formed by the first and second rings, such that the first and second rings can be secured about penises having a variety of diameters. The first and second rings are secured on opposing ends of a penis, wherein blood is introduced and trapped therebetween via the first and second rings. Pressure caused by pressing on the scrotum area increases blood flow within the penis creating small tears that promote cell growth and cell division to repair the tissue, thereby increasing the size of the penis.

### CROSS REFERENCE TO RELATED APPLICATIONS

This application is a continuation-in-part and claims the benefit of U.S. Provisional Application No. 62/045,113 filed on Sep. 3, 2014, U.S. application Ser. No. 14/843,466 filed on Sep. 2, 2015 and also claims the benefit of U.S. Provisional Application No. 62/558,617 filed on Sep. 14, 2017. The above identified patent applications are herein incorporated by reference in their entirety to provide continuity of disclosure.

### BACKGROUND OF THE INVENTION

The present invention relates to a penis enhancement system and a method for using the same. More particularly, the present invention pertains to a penis enhancement apparatus comprising a pair of rings configured to be positioned around opposing ends of a penis in order to increase and isolate blood flow to promote cell growth therebetween. The present invention can be used to enlarge, extend, and enhance the length and girth of a user's penis.

Engaging in intimate acts with another can be embarrassing and uncomfortable for many men if those men do not feel confident about the size, appearance, or girth of one's penis. Some men may resort to taking various drugs in order to increase the size of one's penis. This frequently leads to harmful and dangerous side effects for the user, such as damaged blood vessels and an inability to achieve or maintain an erection. Additionally, other men may use various devices to increase the size of one's penis, however these devices can damage the blood vessels in the penis and injure the user. Therefore, there exists a need for an apparatus that safely and conveniently increases the size of one's penis.

In light of the devices disclosed in the known art, it is submitted that the present invention substantially diverges in design elements from the known art and consequently it is clear that there is a need in the art for an improvement to existing penis enhancement systems. In this regard, the instant invention substantially fulfills these needs.

### SUMMARY OF THE INVENTION

In view of the foregoing disadvantages inherent in the known types of penis enhancement systems now present in the known art, the present invention provides a penis enhancement system wherein the same can be utilized for providing convenience for the user when enlarging one's penis.

It is therefore an object of the present invention to provide a new and improved penis enhancement system that has all of the advantages of the known art and none of the disadvantages.

It is another object of the present invention to provide a penis enhancement system that can be utilized before engaging in sexual activity.

Another object of the present invention is to provide a penis enhancement system comprising a pair of rings, wherein each ring is adjustable in diameter so as to accommodate the penis size of the user.

Yet another object of the present invention is to provide a penis enhancement system having a method of use intended to promote the physiological process of cell growth or hypertrophy and cell division known as hyperplasia in order to enlarge the girth and length of the penis.

Another object of the present invention is to provide a penis enhancement system having a method of user that stimulates cell growth in the penis of the user.

Another object of the present invention is to provide a penis enhancement system that is flexible.

Another object of the present invention is to provide a penis enhancement system having a cord lock device for adjusting the diameter of each of the first and second rings.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows an exploded view of an embodiment of the first ring of the penis enhancement system.

FIG. 1B shows an exploded view of an embodiment of the second ring of the penis enhancement system.

FIG. 2 shows a perspective view of the first ring of an embodiment of the penis enhancement system.

FIG. 3 shows a view of the first ring applied to the penis.

FIG. 4 shows a view of the first ring and the second ring applied to the penis.

### DETAILED DESCRIPTION OF THE INVENTION

Reference is made herein to the attached drawings. Like reference numerals are used throughout the drawings to depict like or similar elements of the penis enhancement system. For the purposes of presenting a brief and clear description of the present invention, the preferred embodiment will be discussed as used for causing small tears or micro-tears in blood vessels and the tissue of the penis in order to promote new cell growth therein, thus increasing the size of one's penis. The preferred embodiment further includes a cord lock system similar to a drawstring, however alternative adjustable fastening systems are contemplated. The figures are intended for representative purposes only and should not be considered to be limiting in any respect.

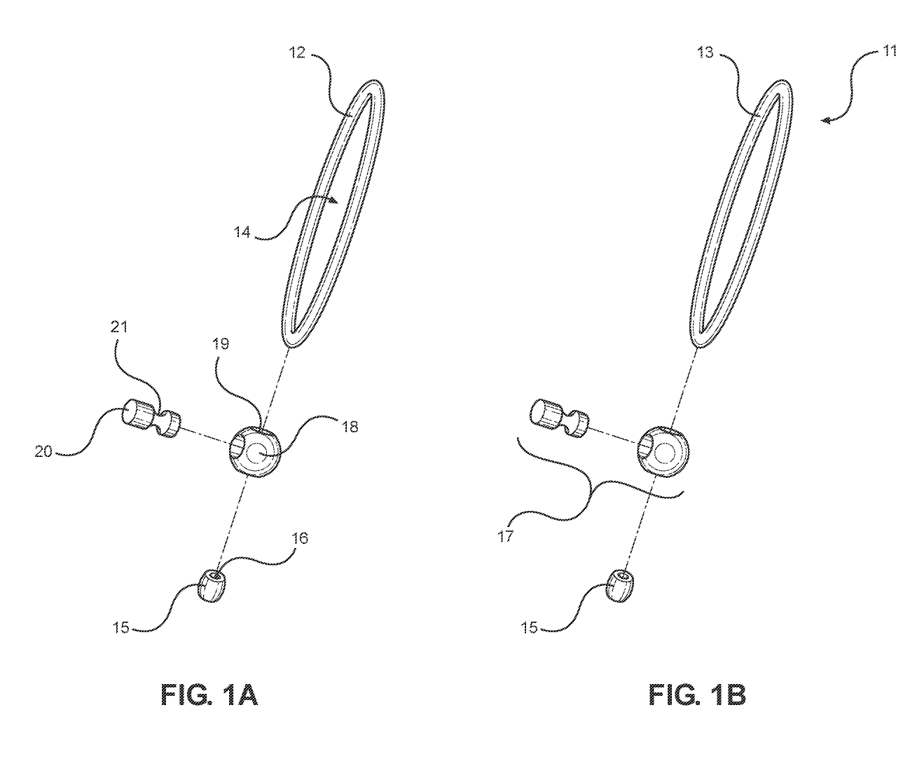

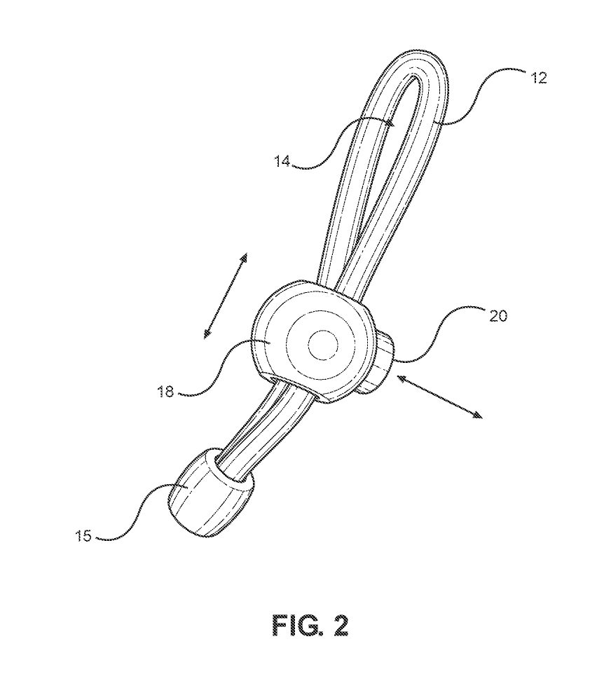

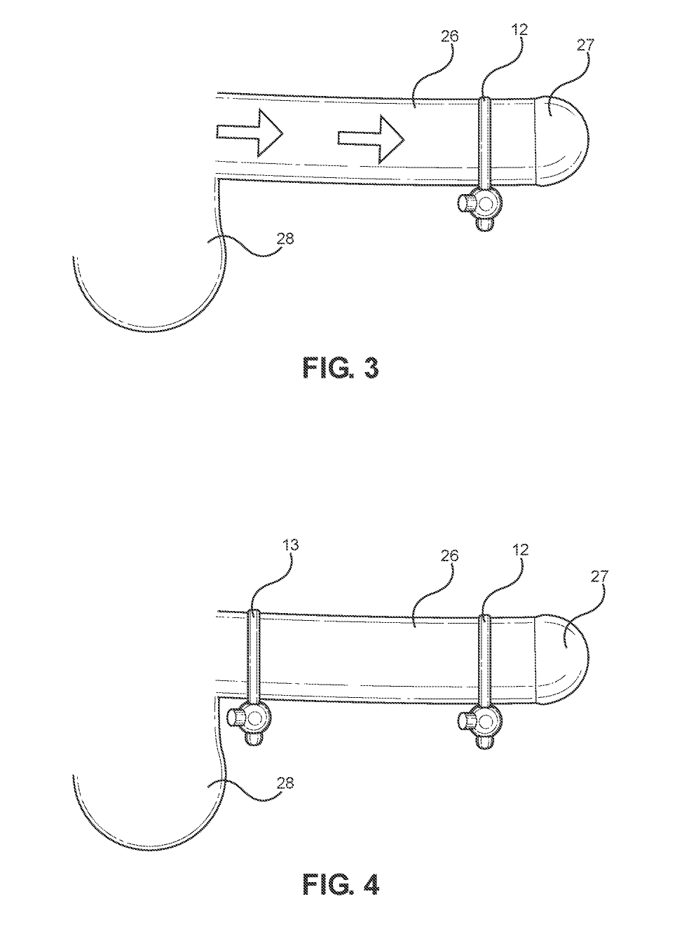

Referring now to FIGS. 1A, 1B, and 2, there is shown an exploded view of an embodiment of the first ring of the penis enhancement system, an exploded view of an embodiment of the second ring of the penis enhancement system, and a perspective view of the first ring of an embodiment of the penis enhancement system, respectively. The penis enhancement system 11 is configured to be disposed on a penis having a tip (as shown in FIG. 3, 27) and a base, wherein the tip of the penis comprises the glans. The glans extends into a shaft (as shown in FIG. 3, 26) of the penis and the base, which in turn extends into a scrotum (as shown in FIG. 3, 28). It should be noted that the penis contains several columns made of tissue. The corpus cavernosum comprises two columns that extend along each side of the penis, wherein blood fills these columns to cause an erection. The corpus spongiosum consists of a column of sponge-like tissue that extends from the base of the penis to the glans of the penis.

In the illustrated embodiment of FIGS. 1A and 1B, the penis enhancement system 11 comprises a first ring 12 and a second ring 13, wherein each ring comprises a flexible band. In the illustrated embodiment, the flexible band comprises latex, however other elastic materials are contemplated. The rings 12, 13 are circular in shape and comprise an opening 14 configured to receive the penis therethrough. Each of the first and second rings 12, 13 is configured to be placed onto the shaft of the penis, wherein the rings 12, 13 lie flush against the shaft, thereby circumscribing a circumference thereof. In the illustrated embodiment, each of the first and second rings 12, 13 comprise a toroidal shape configured to provide a smaller surface area in contact with the penis, thereby increasing the pressure generated thereby. In some embodiments, the flexible band comprises a planar structure having an interior surface configured to rest flush against the penis when secured thereto. In some embodiments, the interior surface further comprises a plurality of protrusions, the plurality of protrusions configured to increase the pressure applied to the tissue of the shaft, thus contributing to the production of small tears in the tissue of the penis.

Each ring 12, 13 of the penis enhancement system 11 further comprises a cord lock 17, wherein the cord lock 17 comprises a housing 18 having a first hole 19 therein. The first hole 19 is configured to receive one of the first and second rings 12, 13 therethrough. A clamp member 20 is spring-biased within the housing 18, wherein the clamp member 20 further comprises a second hole 21 therein, the second hole 21 configured to receive one of the first and second rings 12, 13 therethrough. The clamp member 20 is configured to selectively move between an extended position and a retracted position, wherein the clamp member 20 is spring-biased towards the extended position. The clamp member 20 is configured such that when the clamp member 20 is in the retracted position the first hole 19 and the second hole 21 align, allowing the housing 18 to slide along each of the first and second rings 12, 13 to adjust a diameter of the opening 14 thereof. In this way, the user can alter the diameter of each of the first and second rings 12, 13 to a desired size, similar to a drawstring.

The penis enhancement system 11 further comprises a pair of beads 15, each having an aperture 16 therein. The aperture 16 is configured to receive one of the first and second rings 12, 13 therethrough, such that each of the pair of beads 15 can be affixed thereto. In some embodiments, the bead 15 is affixed to each ring 12, 13 via an adhesive, while in other embodiments, each ring 12, 13 can be secured within the aperture 16 via friction fit. The pair of beads 15 are configured to provide a gripping surface to the user when sliding the housing 18 along each ring 12, 13. In some embodiments, each of the pair of beads 15 comprise a diameter greater than that of the first and second holes 19, 21, thereby preventing the housing 18 from moving past each of the pair of beads 15. In this way, a maximum diameter of the opening 14 can be defined by the pair of beads 15, while also retaining the cord lock 17 on each of the first and second rings 12, 13.

Referring now to FIGS. 3 and 4, there is shown a view of the first ring applied to the penis and a view of the first ring and the second ring applied to the penis, respectively. In one exemplary use, the method of using the penis enhancement system comprises providing a first ring 12 and a second ring 13, wherein each of the first and second rings 12, 13 comprise an opening configured to receive a penis therethrough. The user can then adjust the diameter of the opening by actuating a spring-biased clamp member disposed within a housing, and sliding the housing along one of the first and second rings 12, 13. Once adjusted to the correct diameter, the user places the first ring 12 adjacent to the tip 27 of the penis about the shaft 26 thereof and places the second ring 13 onto a base of the penis opposite the tip 27 about the shaft 26 thereof. The user then squeezes the scrotum 28 disposed adjacent to the base to induce blood flow into the shaft between the first and second rings 12, 13.

Before use, the user can adjust the size of the first ring 12 and the second ring 13 in order to provide a desired diameter to fit the size of the penis of the user, so as to allow blood to be contained therebetween. The penis should preferably be semi-erect when using the penis enhancement system. In the illustrated embodiment, the clamp member is actuated and the housing is moved along the ring to adjust the diameter thereof. Then the user can proceed to place the first ring 12 on the shaft 26 of the penis, adjacent to the tip 27. Once the first ring 12 is placed below the glans, the first ring 12 causes pressure to be exerted onto the shaft, thus preventing blood from moving beyond the first ring 12. The internal pressure and stress caused by blood accumulation within the penis creates small tears or micro-tears to the corpus cavernosum and corpus spongiosum tissue.

The second ring 13, once applied, isolates blood flow in the penis between the first ring 12 and the second ring 13. The second ring 13 can further prevent blood flow from returning to the body and further increases the volume of blood in the penis in order to cause the small tears created in the tissue of the penis. The second ring 13 further allows blood to remain in the shaft 26 to promote cell growth and cell division for healing the small tears in the penis. The user can then further increase stress and pressure onto the tissue of the penis by applying additional external pressure via pressing or squeezing the scrotum 28 area. This additional pressure causes high tension overload within the tissues of the penis causing further micro-tears in the tissues of the shaft 26, which in turn causes the body to respond by increasing blood flow to the area in which the micro-tears are located in order to repair the micro-tears. As the blood flows to the small tears in the tissue of the penis, the tissues of the penis undergo cell growth known as hypertrophy and cell division known as hyperplasia. These physiological processes of hypertrophy and hyperplasia result in permanent increases in both length and girth of the shaft 26.

The penis enhancement system can be used every day, every other day, or as needed. The amount of time the penis enhancement system can be used may vary in correlation to the amount in which the user wishes to increase the size of his penis. The penis enhancement apparatus can be utilized in various increments being five, ten, fifteen, twenty, twenty-five minutes or more, if desired or needed by the user. The penis enhancement system can be made in various sizes and colors in order to accommodate the needs of the user.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A penis enhancement system, comprising:
a first ring and a second ring, wherein each of the first ring and the second ring comprise a flexible band and an opening configured to receive a penis therethrough;
a pair of beads each affixed to one of the first and second rings;
wherein each of the pair of beads comprises an aperture therein, the aperture configured to receive one of the first and second rings therethrough;
a pair of cord locks each slidably disposed about one of the first and second rings;
wherein each of the pair of cord locks comprise a housing having a first hole therein;
a spring-biased clamp member disposed within the housing, the clamp member configured to selectively move between an extended position and a retracted position;
wherein the clamp member further comprises a second hole therein;
wherein the first and second holes are aligned when the clamp member is in the retracted position.

2) The penis enhancement system of claim 1, wherein the pair of beads comprise a greater diameter than a diameter of each of the first and second holes.

3) The penis enhancement system of claim 1, wherein each of the first ring and the second ring are composed of latex.

4) The penis enhancement system of claim 1, wherein each of the first ring and the second ring comprise a toroidal shape.

5) The penis enhancement system of claim 1, wherein each of the first and second rings are adjustable in diameter via sliding the cord lock therealong.

6) A method of enlarging the penis comprising:
providing a first ring and a second ring, wherein each of the first ring and the second ring comprise an opening configured to receive a penis therethrough;
wherein each of the first and second rings further comprise a cord lock slidably disposed thereon, the cord lock having a clamp member therein, wherein the cord lock is configured to slide along the first and second rings when the clamp member is actuated;
actuating the clamp member to slide the cord lock along each of the first and second rings until the openings comprise a desired diameter to fit a penis;
placing the first ring near a tip of a shaft of the penis, wherein the placement of the first ring constrains blood flow within the penis, thereby causing pressure to one or more tissues of the penis, creating a plurality of small tears in the one or more tissues of the penis.

7) The method of enlarging the penis of claim 6, further comprising:
placing the second ring onto a base of the shaft of the penis, wherein the placement of the second ring isolates blood between the first ring and the second ring, the isolated blood causing a plurality of small tears in the one or more tissues of the penis;

8) The method of enlarging the penis of claim 6, further comprising squeezing a scrotum to pump the blood of the penis into the shaft of the penis to cause the plurality of small tears in the one or more tissues of the penis, wherein the creation of the plurality of small tears in the one or more tissues of the penis induces physiological processes of cell growth and cell division therein, thereby causing a permanent increase in length and girth of the penis.
